(19) United States
(12) Patent Application Publication
(10) Pub. No.: US 2026/0034288 A1
(43) Pub. Date: Feb. 5, 2026

(54) SYSTEM FOR INLINE PRODUCTION OF PERITONEAL DIALYSIS FLUID

(71) Applicant: Mozarc Medical US LLC, Minneapolis, MN (US)

(72) Inventors: Claudio Marella, Mirandola (IT); Mariachiara Ricci, Mirandola (IT); Marta Prati, Mirandola (IT); Michele Passerini, Mirandola (IT); Roberta Leonardi, Mirandola (IT); Andrea Veratti, Mirandola (IT); Giuliano Giganti, Mirandola (IT); Stefania Rinaldi, Mirandola (IT)

(21) Appl. No.: 19/285,919

(22) Filed: Jul. 30, 2025

Related U.S. Application Data
(60) Provisional application No. 63/678,401, filed on Aug. 1, 2024.

(51) Int. Cl. A61M 1/28; A61M 1/14

(52) U.S. Cl. CPC A61M 1/287; A61M 1/155; A61M 1/159; A61M 2205/3324; A61M 2205/3327; A61M 2205/3368

(57) ABSTRACT

Systems, components, and methods are provided for a flexible, efficient system capable of producing customized dialysis fluid at the point of care. By enabling precise control over the solution's composition, the system significantly the customization, safety, and effectiveness of dialysis therapy. The placement of sensors and feedback mechanisms ensures accurate monitoring and adjustment of the solution's composition for dialysis fluid for patient treatment.

### CROSS REFERENCE

This application claims the benefit of and priority to U.S. Provisional Patent Application No. 63/678,401, filed Aug. 1, 2024, the entire content of each of which is incorporated by reference herein.

### FIELD

The present disclosure relates to dialysis systems. More particularly, this disclosure relates to systems and methods for inline production of fluid solutions in dialysis systems such as, but not limited to, peritoneal dialysis systems.

### BACKGROUND

Peritoneal dialysis is a common treatment for patients with renal failure, wherein a dialysis solution is used to remove waste products from the blood. The composition and quality of this dialysis fluid can impact the effectiveness and safety of the treatment. The preparation of peritoneal dialysis fluid involves pre-mixed solutions.

### SUMMARY

The problem to be solved is production of peritoneal dialysis fluid for using integrated sensor and control technologies, on-site at the location of therapy and/or on-demand, to create a suitable peritoneal dialysis solution.

The first aspect relates to a system for inline production of peritoneal dialysis fluid that can include a mixing section designed to combine concentrate and water. This system is further equipped with a sensor for measuring the conductivity of the produced fluid and a controller programmed to adjust the proportions of concentrate and water based on the conductivity measurements, thereby forming a peritoneal dialysis solution.

In any embodiment, the controller can be further programmed to perform real-time adjustments of the concentrate and water proportions during the production of the peritoneal dialysis solution, enhancing the adaptability and precision of the system.

In any embodiment, the sensor utilized within the system can be an electrical conductivity sensor. The sensor can measure the fluid's conductivity for proper formulation of the dialysis solution.

In any embodiment, the mixing unit of the system can include separate channels for the concentrate and water. This can allow for the controlled flow of each component, contributing to a desired quality of the produced dialysis solution.

In any embodiment, the system can further include a storage unit for the prepared dialysis solution. This addition can provide a means for storing the solution until it is needed.

In any embodiment, the controller can be programmed to store historical data of conductivity measurements and adjustments made. This capability provides the system a reference to past operations for improved accuracy and efficiency in future solution preparations.

In any embodiment, a user interface can be included for displaying the current conductivity measurements and adjustments made by the controller.

In any embodiment, the concentrate used in the system can comprise at least one of glucose, electrolytes, and bicarbonate. This composition flexibility can provide for a range of dialysis solutions tailored to specific patient needs.

In any embodiment, the water utilized in the system can be purified or sterilized water.

In any embodiment, the system can further include one or more negative feedback loops for enhanced precision in solution preparation. These feedback loops incorporate additional sensors for measuring other parameters of the solution for monitoring and control of the solution's quality.

In any embodiment, each negative feedback loop can include additional sensors for measuring other parameters of the solution, such as temperature and osmolality. These measurements can assist in maintaining a desired composition of the dialysis solution.

In any embodiment, the system is configured for use in both clinical and home settings. This versatility allows the system to meet the diverse needs of patients, providing them with high-quality dialysis treatment.

In some embodiments, a system for inline production of peritoneal dialysis fluid includes a mixing section for combining concentrate and water, an electrolyte sensor for measuring a produced fluid, and a controller programmed to adjust proportions of concentrate and water based on the measurements by the electrolyte sensor, thereby forming a peritoneal dialysis solution.

In some embodiments, the controller is further programmed to perform real-time adjustments of the concentrate and water proportions during production of the peritoneal dialysis solution.

In some embodiments, the sensor is an electrical conductivity sensor.

In some embodiments, the mixing section includes separate channels for the concentrate and the water.

In some embodiments, the mixing section is a static mixer.

In some embodiments, the controller is programmed to store historical data of conductivity measurements and adjustments made.

In some embodiments, the system includes a user interface for displaying current conductivity measurements and adjustments made by the controller.

In some embodiments, the concentrate includes at least one of: dextrose, electrolytes, and bicarbonate.

In some embodiments, the water is purified or sterilized water.

In some embodiments, the system includes one or more negative feedback loops for enhanced precision in solution preparation.

In some embodiments, each negative feedback loop includes additional sensors for measuring other parameters of the peritoneal dialysis solution.

In some embodiments, the other parameters include pH, temperature, and osmolality.

In some embodiments, the system is configured for use in both clinical and home settings.

In some embodiments, a system for inline production of peritoneal dialysis fluid, includes a mixing unit for combining concentrate and water, a sensor for measuring conductivity of produced fluid, and a controller programmed to adjust proportions of concentrate and water based on the conductivity measurements, thereby forming a dialysis solution.

In some embodiments, the controller is further programmed to perform real-time adjustments of the concentrate and water proportions during production of the dialysis solution.

In some embodiments, the mixing unit includes separate channels for the concentrate and the water.

In some embodiments, the system includes a storage unit for prepared dialysis solution.

In some embodiments, the system includes a user interface for displaying current conductivity measurements and adjustments made by the controller.

In some embodiments, the concentrate includes at least one of: glucose, electrolytes, and bicarbonate.

In some embodiments, the system includes one or more negative feedback loops for enhanced precision in solution preparation.

The features disclosed as being part of the first aspect can be in the first aspect, either alone or in combination, or follow any arrangement or permutation of any one or more of the described elements. Similarly, any features disclosed as being part of the first aspect can be in the first aspect, either alone or in combination, or follow any arrangement or permutation of any one or more of the described elements.

The second aspect of the system for inline production of peritoneal dialysis fluid mirrors the innovative features of the first aspect but is presented as a distinct configuration. This aspect also emphasizes the system's capability to adjust the proportions of concentrate and water based on conductivity measurements, forming a dialysis solution tailored to the needs of the patient.

In any embodiment, a system for inline production of peritoneal dialysis fluid can include a mixing unit designed to combine concentrate and water. This system is equipped with a sensor for measuring the conductivity of the produced fluid and a controller programmed to adjust the proportions of concentrate and water based on the conductivity measurements, thereby forming a dialysis solution.

In any embodiment, the controller can be further programmed to perform real-time adjustments of the concentrate and water proportions during the production of the dialysis solution. This feature allows for dynamic response to changes in fluid composition, ensuring the dialysis solution is consistently formulated to meet specific requirements.

In any embodiment, the sensor utilized within the system can be an electrical conductivity sensor.

In any embodiment, the mixing unit can include separate channels for the concentrate and water.

In any embodiment, the system can further include a storage unit for the prepared dialysis solution.

In any embodiment, the controller is programmed to store historical data of conductivity measurements and adjustments made.

In any embodiment, a user interface can be included for displaying the current conductivity measurements and adjustments made by the controller.

In any embodiment, the concentrate used in the system can comprise at least one of glucose, electrolytes, and bicarbonate.

In any embodiment, the water utilized in the system can be purified or sterilized water.

In any embodiment, the system can further include one or more negative feedback

loops for enhanced precision in solution preparation.

In any embodiment, each negative feedback loop can include additional sensors for measuring other parameters of the solution, such as temperature or osmolality that can be adjusted based on one or more temperature sensors.

The features disclosed as being part of the second aspect can be in the second aspect, either alone or in combination, or follow any arrangement or permutation of any one or more of the described elements. Similarly, any features disclosed as being part of the second aspect can be in the first aspect, either alone or in combination, or follow any arrangement or permutation of any one or more of the described elements.

### BRIEF DESCRIPTION OF THE DRAWINGS

FIG. 1 illustrates a system configuration where a precise volumetric pump is used to ensure the correct flowrate of purified water into the mixing section.

FIG. 2 illustrates a system setup incorporating a dedicated sensor for flowrate control.

FIG. 3 illustrates scales placed under the water reservoir are shown.

FIG. 4 illustrates a system where scales are utilized under a bag collecting the generated peritoneal dialysis fluid (PDF).

FIG. 5 illustrates a configuration featuring both electrolyte and osmotic agent sensors positioned downstream of the concentrate bags and the mixing component.

FIG. 6 illustrates a system where sensors are placed immediately after the corresponding concentrate bags but before the mixing component.

FIG. 7 illustrates a system without strict positioning constraints for the sensors, allowing for flexibility in the system layout.

FIG. 8 illustrates a flow chart of a negative feedback loop.

### DETAILED DESCRIPTION

The preparation of peritoneal dialysis fluid (“PDF”) can lead to variability in the composition and quality of the solution. These methods often lack the flexibility to adjust the fluid composition in real-time based on specific patient needs, on-site, and/or on-demand. The preparation of dialysis fluid requires precise control over the concentration of various components, such as glucose, electrolytes, and bicarbonate. Any deviation from the desired concentration beyond an acceptable tolerance range can impact the treatment's effectiveness and pose risks to the patient's health. Typically, pre-determined proportions of concentrate and water are utilized, without the capability to dynamically adjust these ratios during the fluid preparation process. This can lead to inconsistencies in the final dialysis fluid composition, especially if the concentrate or water source varies in quality or concentration. Furthermore, these is a lack of real-time feedback, thereby limiting the ability to make adjustments as needed. The inability to fine-tune the solution's composition in real-time poses a constraint in the real-time preparation of solutions for treatment purposes. This limitation constrains the ability to create the proper solutions at the site of preparation. The inability to adjust the dialysis solution's composition reverts to reliance on pre-mixed solutions.

Various embodiments of the present disclosure relate to systems, devices, apparatuses, and methods for the production of peritoneal dialysis fluid on-site and/or on-demand. The embodiments can be capable of accurately measuring the fluid's properties, adjusting its composition in real-time, and ensuring that the final product meets the standards for effective dialysis treatment. The embodiments can utilize sensors, feedback loops, and control algorithms to produce high-quality dialysis fluid efficiently and consistently. The embodiments thereby improves the production of peritoneal dialysis fluid through adaptability, precision, and enhanced user interaction, offering significant benefits to both patients and healthcare providers. The embodiments in the present disclosure provide reliable and precise dialysis fluid preparation methods, ensuring optimal treatment outcomes using a system that reconstitutes concentrates on-site and/or on-demand. It is to be appreciated that the concentrates can be reconstituted from a powder form. The embodiments also enable on-demand production of dialysis fluid, reducing the need for storage and handling of large volumes of pre-packaged fluid.

The systems and methods of the present embodiments are provided for use in the medical field, specifically for renal care and dialysis treatment. The systems and methods are used in the preparation of peritoneal dialysis fluid used in both clinical and home settings. The systems and methods can produce dialysis fluid inline efficiently, accurately, and safely for patients undergoing peritoneal dialysis treatment. The embodiments relate to medical devices and methods, specifically to a system and method for producing peritoneal dialysis fluid on-demand with enhanced precision and customization capabilities. The systems and methods can provide reliable and precise dialysis fluid preparation methods, ensuring optimal treatment outcomes using a system the reconstitutes concentrates on-site.

The various embodiments of the present disclosure relate to customization of the dialysis solution to meet individual patient requirements, enhancing therapy effectiveness. The embodiments also relate to improved patient safety through the use of freshly prepared, precisely composed dialysis fluid.

Unless defined otherwise, all technical and scientific terms used have the same meaning as commonly understood by one of ordinary skill in the art.

The articles “a” and “an” are used to refer to one to over one (i.e., to at least one) of the grammatical object of the article. For example, “an element” means one element or over one element.

The term “comprising” includes, but is not limited to, whatever follows the word “comprising.” Use of the term indicates the listed elements are required or mandatory but that other elements are optional and may be present.

The terms “connected,” “connection,” to “connect,” or “connectable” refers to the ability of forming physical contact between two components or parts. The connection need not be permanent.

The term “consisting of” includes and is limited to whatever follows the phrase “consisting of.” The phrase indicates the limited elements are required or mandatory and that no other elements may be present.

The term “consisting essentially of” includes whatever follows the term “consisting essentially of” and additional elements, structures, acts, or features that do not affect the basic operation of the apparatus, structure or method described.

The terms “control,” “controlling,” or “controls” can refer to the ability of one component to direct the actions of a second component.

A “control system” can be a combination of components that act together to maintain a system to a desired set of performance specifications. The control system can use processors, memory and computer components configured to interoperate to maintain the desired performance specifications. The control system can also include fluid or gas control components, and solute control components as known within the art to maintain performance specifications.

A “controller” can refer to a device or set of devices responsible for managing and regulating the operations of other components within a system. The controller can be programmed to perform operations based on instructions executed by a processor of the controller.

A “concentrate” typically means a substance made by removing or reducing the diluting agent; in the context of dialysis, it usually refers to a solution concentrated with substances like glucose, electrolytes, or bicarbonate. A concentrate can be a high concentration solution prepared by adding solvent to a powdered substance.

The term “conductivity measurements” involve determining the ability of a fluid to conduct an electrical current, often used to assess the concentration of ions or the salinity of the solution.

A “dialysis solution” is a fluid used in dialysis, typically comprising purified water, electrolytes, and other additives, to facilitate the removal of waste products from the blood.

An “electrical conductivity sensor” is a device used to measure the electrical conductivity in a solution, which is indicative of its ion concentration.

A “feedback loop” refers to a system mechanism where a portion of the output is returned or fed back to the input to control its further output. A “fluid passage” is a conduit or pathway through which fluid or gas can move.

The term “fluidly connectable,” “fluid connection,” “fluidly connectable,” “fluidically engage”, or “fluidically coupled” refers to the ability of providing for the passage of fluid, gas, or combination thereof, from one point to another point. The ability of providing such passage can be any connection, fastening, or forming between two points to permit the flow of fluid, gas, or combinations thereof. The two points can be within or between any one or more of compartments of any type, modules, systems, components, and rechargers.

The term “fluidly connected” refers to a particular state such that the passage of fluid, gas, or combination thereof, is provided from one point to another point. The connection state can also include an unconnected state, such that the two points are disconnected from each other to discontinue flow. It will be further understood that the two “fluidly connectable” points, as defined above, can form a “fluidly connected” state. The two points can be within or between any one or more of compartments, modules, systems, components, and rechargers, all of any type.

The term “dextrose” is a simple sugar and an important energy source in living organisms and a component of many carbohydrates.

The term “historical data” refers to previously recorded information or data points, used in this context to track past conductivity measurements and adjustments.

The term “inline production” means the process of creating or manufacturing a mixed fluid within a line of operation using a plurality of simultaneous streams of solutions, typically without needing to divert materials or components to a separate area or mixing container.

A “mixing section” or a “mixing unit” refers to a tubing or portion thereof, a flow path, device, apparatus, or component used to combine or blend different substances, such as concentrate and water, to form a homogenous mixture.

The term “negative feedback loop” is a control mechanism where the system's output is fed back into the system in a manner that tends to reduce fluctuations or deviations from a set point, thereby stabilizing the system.

The term “osmolality” measures the concentration of solutes per unit of solvent in a solution, often used to assess the osmotic pressure of a body fluid.

The term “pH” is a scale used to specify the acidity or basicity of an aqueous solution, where lower values indicate acidity and higher values indicate alkalinity.

The term “purified water” is water that has been filtered, processed, or combinations thereof, to remove impurities and make it suitable for use.

The term “real-time” refers to processing data or performing actions almost immediately after data is input or an instruction is received, without noticeable delay.

The term “storage unit” in this context likely refers to a container or area designated for holding and preserving the prepared dialysis solution until it is needed.

The term “sterilized water” is water that has been treated to eliminate all forms of bacteria, viruses, and other microorganisms to make it suitable for medical use.

The term “temperature” in the context of this system would refer to the degree of heat present in the dialysis solution, which can affect its properties and effectiveness.

The term “touch screen” is an electronic visual display that can detect the presence and location of a touch within the display area, used here for inputting and displaying data.

The term “user interface” refers to the means by which the user interacts with a machine, equipment, or software, often involving a display screen, keyboard, or other control mechanism.

### Inline Production of Peritoneal Dialysis Fluid

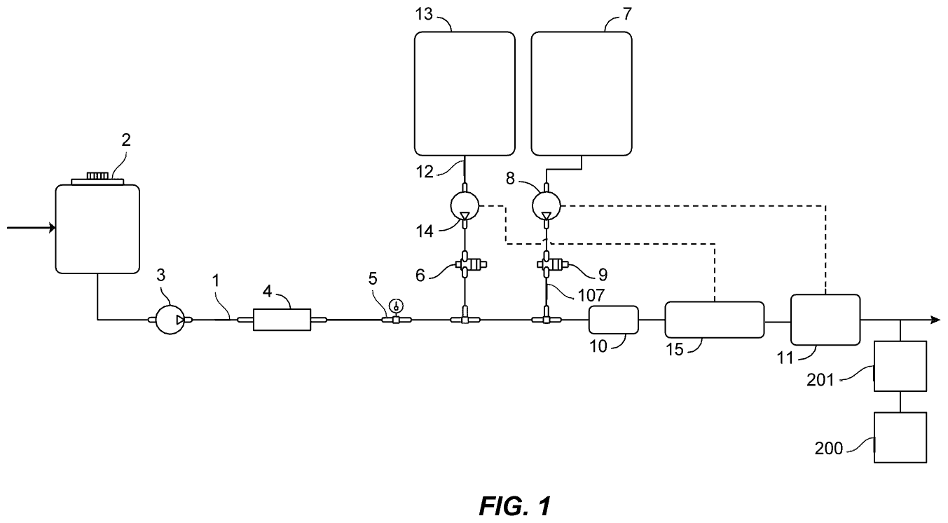

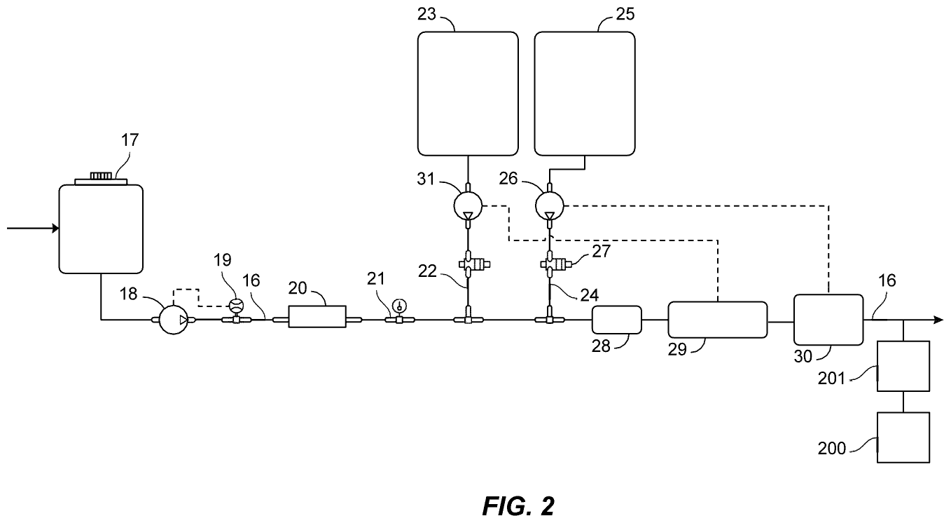

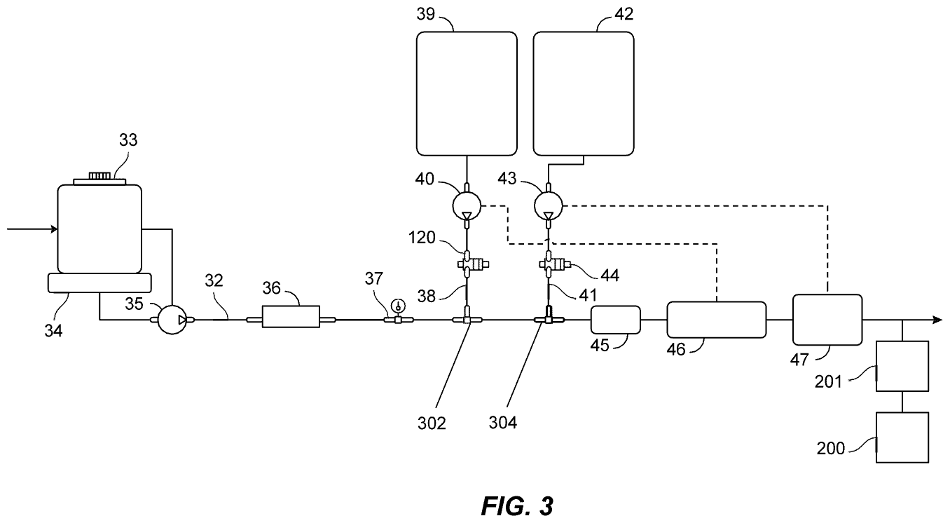

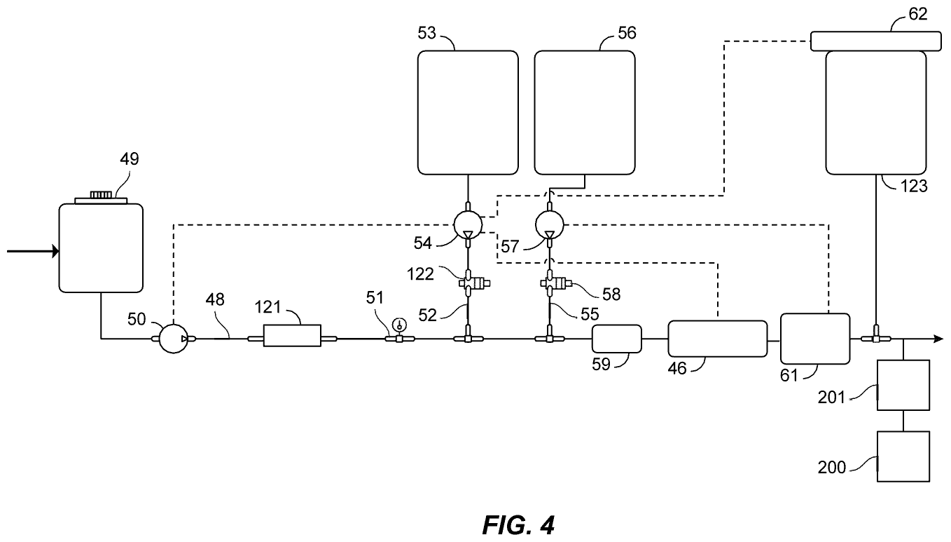

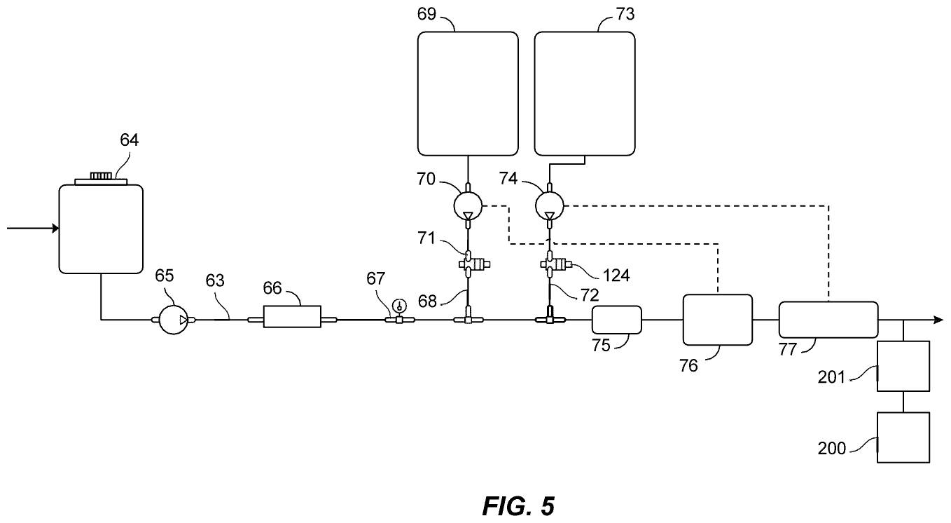

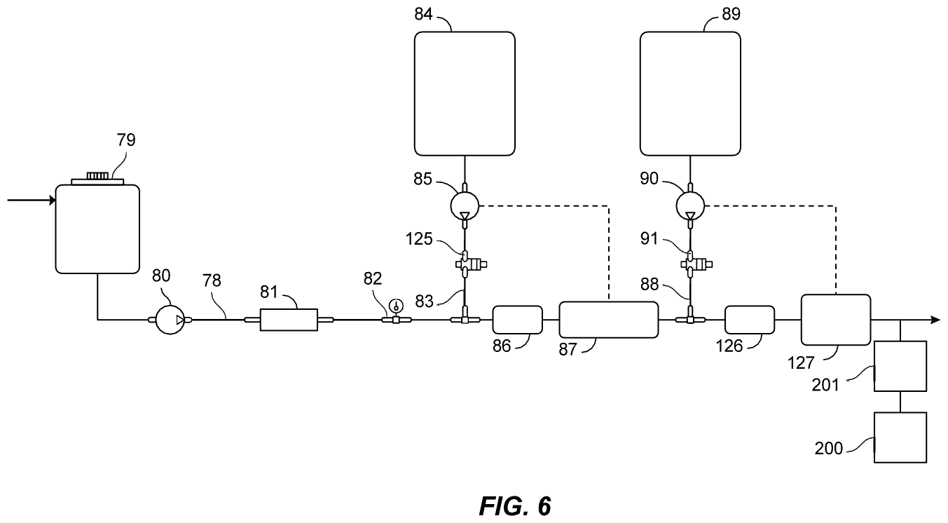

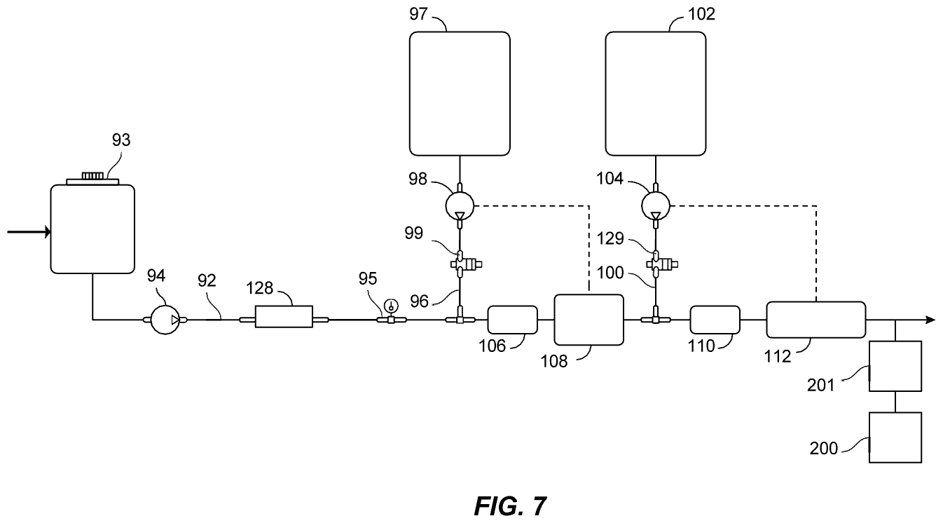

The systems and methods provide for an inline and on-demand production of peritoneal dialysis fluid (PDF), which enhances the efficiency and safety of dialysis treatment. The system operates by proportioning simultaneous streams of purified water and dialysate chemical constituents, either in powder or liquid form, to create the dialysis solution. For powdered constituents, a reconstitution process with water is employed to form liquid concentrates. The system includes at least two types of concentrate bags: one for electrolytes and another for the osmotic agent, ensuring a comprehensive approach to solution composition. The PDF's production is controlled through a series of one or more feedback loops and sensors that are used to accurately meter the concentrates using a set flowrate of the purified water. In some embodiments, the set flowrate can be controlled using a precise volumetric pump (FIG. 1) or a flowrate control system that relies on either a dedicated sensor (FIG. 2) or scale-derived flowrate calculations, with scales placed under the water reservoir (FIG. 3) or the bag collecting the PDF (FIG. 4). It is to be appreciated that the bag collecting the PDF (FIG. 4) can alternatively be hung from a scale or load cell (as opposed to being placed on top of the scale). This precise control extends to the metering of concentrates, facilitated by pumps in a control loop with sensors specifically for electrolyte and osmotic agent concentrations. Further ensuring the homogeneity and correct composition of the PDF, the system may incorporate additional mixing components within the fluid path, with specific figures (FIG. 1 and FIG. 5) positioning electrolyte and osmotic agent sensors downstream of the concentrate bags and mixing component. FIG. 6 and FIG. 7 are non-limiting embodiments showing sensors positioned immediately after the concentrate bags and the mixing component, allowing for immediate adjustment based on sensor feedback. The resulting PDF can be collected into disposable bags for use in PD therapy, embodying a method that allows for the on-demand production of dialysis fluid directly at the point of care. This method not only reduces preparation time but also significantly improves patient safety by ensuring the correct composition and homogeneity of the dialysis solution throughout the preparation process.

FIG. 1 illustrates a system for performing inline production of a solution, according to some embodiments. The system configuration includes a precise volumetric pump is used to ensure the correct flowrate of purified water into the mixing section. The concentrate and water are combined in a controlled manner, with conductivity and osmotic agent measurements guiding the adjustment of their proportions to form the peritoneal dialysis solution. The system is structured around a main line 1, which serves as the conduit through which the various components of the dialysis fluid are combined and processed. At the beginning of the process, a purified water reservoir 2 is connected to the main line 1 via a pump 3. This pump 3 is responsible for delivering purified water from the reservoir 2 into the main line 1 for controlled flow of water into the system. Following the pump 3, the water passes through a heater 4, where the temperature of the water is adjusted to match the optimal conditions for dialysis fluid preparation. The precise temperature of the water is then verified by a temperature sensor 5, ensuring that the water reaches the desired temperature before it mixes with other components. Although shown and described with the pump 3 being upstream of the heater 4, it is to be appreciated that these components can be reversed in order. That is, in some embodiments, the heater 4 can be upstream of the pump 3. In some embodiments, because volume changes with temperature, the placement of the heater 4 upstream of the pump 3 can result in more precise control by accounting for density changes of the water.

Connected to the main line 1, an osmotic agent line 6 is established, which includes osmotic concentrate reservoir 7. The reservoir 7 contains the osmotic agent concentrate, which is used to create the osmotic gradient for the dialysis process. The osmotic agent from the reservoirs 7 is pumped into the osmotic agent line 6 by an osmotic pump 8 for controlled delivery of the osmotic agent into the system. An osmotic agent valve 9 is then used to introduce the osmotic agent into the main line 1, where it meets the pre-heated purified water. Upon the introduction of the osmotic agent into the main line 1, the mixture encounters the first mixer 10. This mixer 10 ensures thorough blending of the osmotic agent with the purified water and electrolytes, creating a preliminary dialysis solution. Immediately following this mixing process, an osmotic agent sensor 11 measures the concentration of the osmotic agent in the solution, providing real-time data on its composition. In some embodiments, downstream of the osmotic agent sensor 11 can be a fluid connection to a drain 200. In some embodiments, if the osmotic agent sensor 11 measures a concentration that is outside of tolerance limits of a threshold, the fluid can be directed to the drain 200 and removed from the system. In some embodiments, the drain 200 can include a valve 201 that can be automatically enabled or disabled to enable or disable, respectively, flow to the drain 200.

Further, the system can include an electrolyte concentrate line 12. In some embodiments, the electrolyte concentrate line 12 can be upstream of the osmotic agent concentrate line 107 (e.g., as shown in FIG. 5 below). In other embodiments, the electrolyte concentrate line 12 can be downstream of the osmotic agent concentrate line 107. The electrolyte concentrate line 12 introduces electrolytes into the main line 1. The electrolyte concentrate, stored in a reservoir 13, is pumped into the main line 1 using an electrolyte pump 14. An electrolyte concentrate valve 6 is then used to introduce the electrolytes into the main line 1, where it meets the pre-heated purified water. This addition of electrolytes can enable matching the physiological conditions required for effective dialysis in the solution. Following the introduction of electrolytes and osmotic agent, mixer 10 can provide the thorough integration of electrolytes and the osmotic agent into the solution, further refining its composition. In some embodiments, the system can include a second mixer (now shown) to provide the thorough integration of electrolytes into the solution, further refining its composition. The final composition of the dialysis fluid is then assessed by an electrolyte sensor 15, positioned downstream of the mixer 10. In some embodiments, the electrolyte sensor 15 can be positioned downstream of a second sensor. This sensor measures the concentration of electrolytes in the solution, providing feedback on its suitability for dialysis treatment. In some embodiments, if the electrolyte sensor 15 measures a concentration that is outside of tolerance limits of a threshold, the fluid can be directed to the drain 200 and removed from the system.

The system utilizes one or more feedback loops to continuously monitor and adjust the composition of the dialysis fluid based on the measured values from both the osmotic agent sensor 11 and the electrolyte sensor 15. These feedback loops ensure that the concentrations of osmotic agents and electrolytes within the dialysis fluid remain within the desired ranges, guaranteeing the production of a dialysis solution that is precisely tailored to meet the therapeutic needs of patients. Once the dialysis fluid's composition is verified to be within the target specifications, it can be directed to storage or delivered directly to the patient for treatment.

FIG. 2 depicts a system setup incorporating a dedicated sensor for flowrate control. The sensor directly influences the pumping system, adjusting the flowrate of purified water to achieve the desired solution composition based on real-time conductivity feedback, targeted flow rates, and combinations thereof. A main line 16 orchestrates the flow of components through the system to produce the final dialysis fluid. The process begins with a purified water reservoir 17, which supplies water for the dialysis solution. Water from this reservoir 17 is pumped by a pump 18 into the main line 16, initiating the solution's formulation. Immediately following the pump 18, a flowmeter 19 is positioned to accurately measure the flowrate of the water, providing data for maintaining the correct volume and speed of water entering the system. This precise control of water flow is important for achieving the desired dilution ratios of the dialysis solution.

After passing through the flowmeter 19, the water is heated by a heater 20 to reach a temperature conducive to the dialysis process, typically mirroring conditions to dissolve and incorporate the dialysis solution components. The temperature of the water is then verified by a temperature sensor 21, ensuring that it has reached the optimal level before mixing with other components. Connected to the main line 16, an electrolyte concentrate line 22 introduces electrolytes into the system. The electrolyte concentrate, stored in a reservoir 23, is delivered into the main line 16 via an electrolyte pump 31. This step is important for replicating the electrolyte composition of natural bodily fluids, facilitating effective waste removal and fluid balance during dialysis. An osmotic agent line 24 introduces the osmotic agent from a concentrate reservoir 25 into the system. An osmotic pump 26 controls the flow of the osmotic agent for creating the osmotic gradient for the dialysis process. An osmotic agent valve 27, when in an opened or flow enabled state, allows passage of the osmotic agent into the main line 16, where it combines with the pre-heated, electrolyte-enriched water.

Downstream in the main line 16, a mixer 28 ensures thorough integration of the osmotic agent and electrolytes with the water, creating a homogenous dialysis solution. Following this mixing process, the solution's composition is assessed by an electrolyte sensor 29 and an osmotic agent sensor 30. These sensors measure the concentrations of electrolytes and osmotic agents, respectively, providing real-time feedback on the solution's suitability for dialysis treatment. In some embodiments, in addition to providing real-time feedback, the concentrates can be metered to provide different inputs of the agents by adjusting a speed of the electrolyte pump 31, the osmotic agent pump 26, or combinations thereof. In some embodiments, flowmeter 19 is used to adjust the flowrate of the pump 18, in combination with the electrolyte pump 31 and the osmotic agent pump 26, to achieve the desired solution.

The system employs one or more feedback loops that connect back to the flowmeter 19, the pump 18, the electrolyte pump 31, and the osmotic agent pump 26, enabling dynamic adjustments to the flowrates of the pumps based on the measured values from the sensors. This feedback mechanism ensures that any deviations from the desired ranges of electrolyte and osmotic agent concentrations are promptly corrected, maintaining the dialysis solution within the optimal composition parameters. Once the dialysis fluid's composition is verified to be within the target specifications by the sensors, it can be directed to storage or immediately used for patient treatment.

FIG. 3 shows an embodiment where scales placed under the water reservoir are shown. These scales provide feedback on the flowrate of water based on weight changes, allowing for precise control over the volume of water entering the mixing section. A main line 32 facilitates the sequential integration of purified water, electrolytes, and osmotic agents to produce the dialysis solution. The process initiates with a purified water reservoir 33 and a scale 34 to determine a weight of the reservoir 33. In some embodiments, the reservoir 33 can be on top of the scale 34, hung from the scale 34, or the like. This scale 34 provides real-time feedback on the weight of the water in the reservoir 33, which, by extension, allows for precise control over the volume of water introduced into the main line 32. The use of weight measurements for controlling water can improve the accuracy of the solution's dilution ratios.

Water from the reservoir 33 is then moved through the system by a pump 35, ensuring a consistent flow towards the heater 36. The heater 36 adjusts the water's temperature to optimal levels for dialysis, which is then verified by a temperature sensor 37. Ensuring the water reaches the correct temperature. Connected is an electrolyte concentrate line 38 that introduces electrolytes into the main line 32. These electrolytes, stored in a reservoir 39, are delivered via an electrolyte pump 40. An electrolyte concentrate valve 120 then merges the electrolyte concentrate with the main line 32, combining it with the pre-tempered, electrolyte-infused water. In some embodiments, the electrolyte concentrate valve 120 can have two states (e.g., on/off or flow enabled/flow disabled). In some embodiments, the electrolyte concentrate valve 120 can include one or more intermediate states in between the on/off states. In the flow enabled state, the electrolyte concentrate is permitted to flow into the main line 32, merging with purified water at the junction. In some embodiments, the merging location can include a static mixer 302.

The static mixer 302 can, in some embodiments, be configured to receive fluids from one or more sources and to mix the fluid together or enhance the solubility of any concentrates within the fluid. For example, the static mixer 302 can be configured to agitate the fluids, the osmotic agents, the electrolytes, the concentrates, or any combination thereof, to increase the mixing of the constituent parts. In some embodiments, the static mixer 302 can include no moving parts. In some embodiments, the static mixer 302 can include no operated parts. In some embodiments, the static mixer 302 is configured to mix the concentrate and the water without any moving parts. For example, in some embodiments, the static mixer 302 can include a turbulence creating component (not shown) that is designed to reside within the static mixer 302 and increase the turbulence within the fluid passing through the static mixer 302. In some embodiments, the turbulence creating component is a static fixture within the static mixer 302. In some embodiments, the turbulence creating component can move within the static mixer 302 as the fluid passes through the static mixer 302. For example, the turbulence creating component can be driven by the fluid flow. In some embodiments, the static structure of the static mixer 302 can create turbulence in the fluid to increase the mixing of the electrolytes from the electrolyte concentration line 38 and the fluid within the main line 32. The static mixer 302 can function to increase the mixing of the fluids at the junction of the electrolyte concentration line 38 and the main line 32.

An osmotic agent line 41 incorporates an osmotic agent from a concentrate reservoir 42 into the system. Controlled by an osmotic pump 43, the osmotic agent creates the osmotic gradient for the dialysis process. An osmotic agent valve 44 then merges the osmotic agent with the main line 32, combining it with the pre-tempered, electrolyte-infused water. In some embodiments, the osmotic agent valve 44 can have two states (e.g., on/off or flow enabled/flow disabled). In some embodiments, the osmotic agent valve 44 can include one or more intermediate states in between the on/off states. In the flow enabled state, the electrolyte concentrate is permitted to flow into the main line 32, merging with purified water at the junction. In some embodiments, the merging location can include a static mixer 304.

The static mixer 304 can, in some embodiments, be configured to receive fluids from one or more sources and to mix the fluid together or enhance the solubility of any concentrates within the fluid. For example, the static mixer 304 can be configured to agitate the fluids, the osmotic agents, the electrolytes, the concentrates, or any combination thereof, to increase the mixing of the constituent parts. In some embodiments, the static mixer 304 can include no moving parts. In some embodiments, the static mixer 304 can include no operated parts. In some embodiments, the static mixer 304 is configured to mix the concentrate and the water without any moving parts For example, in some embodiments, the static mixer 304 can include a turbulence creating component (not shown) that is designed to reside within the static mixer 304 and increase the turbulence within the fluid passing through the static mixer 304. In some embodiments, the turbulence creating component is a static fixture within the static mixer 302. In some embodiments, the turbulence creating component can move within the static mixer 304 as the fluid passes through the static mixer 304. For example, the turbulence creating component can be driven by the fluid flow. In some embodiments, the static structure of the static mixer 304 can create turbulence in the fluid to increase the mixing of the osmotic agent from the osmotic agent line 41 and the fluid within the main line 32. The static mixer 304 can function to increase the mixing of the fluids at the junction of the osmotic agent line 41 and the main line 32.

Downstream in the main line 32, a mixer 45 ensures the thorough blending of the osmotic agent and electrolytes with the water, producing a homogeneous dialysis solution. The composition of this solution is then assessed by an electrolyte sensor 46 and an osmotic agent sensor 47. These sensors provide data on the concentrations of electrolytes and osmotic agents, respectively, offering real-time insights into the solution's composition. In some embodiments, in addition to providing real-time feedback, the concentrates can be metered to provide different inputs of the agents by adjusting a speed of the electrolyte pump 40, the osmotic agent pump 43, or combinations thereof. In some embodiments, scale 34 is used to adjust the flowrate of the pump 35, in combination with the electrolyte pump 40 and the osmotic agent pump 43, to achieve the desired solution. In some embodiments, the mixer 45 can be a static mixer. In some embodiments, the mixer 45 can include a static mixer.

The system employs feedback loops that connect back to the scale 34, the pump 35, the electrolyte pump 40, and the osmotic agent pump 43, enabling adjustments to the flowrates and resulting solution to be made based on the solution's weight. This feedback mechanism ensures that any deviations from the desired concentration ranges of electrolytes and osmotic agents are promptly identified and corrected, maintaining the dialysis solution within optimal composition parameters. Once the composition of the dialysis fluid is confirmed to be within the target specifications by the sensors, it can be directed to storage or immediately utilized for patient treatment. If the solution is outside of the tolerance limits of any of the target specifications, the fluid can be directed to a drain (e.g., as in the drain 200 (FIG. 1) with the valve 201 that can be automatically enabled or disabled to enable or disable, respectively, flow to the drain 200).

FIG. 4 shows a system where scales are utilized with the bag collecting the generated peritoneal dialysis fluid (PDF). In some embodiments, the bag can be placed on the scale. In some embodiments, the bag can be hung from a scale. The weight measurements from the scales inform the system of the flowrate, aiding in the accurate metering of purified water. This setup is focused on leveraging real-time weight measurements to fine-tune the solution's composition. The process begins with a purified water reservoir 49. Water is drawn from this reservoir by a water pump 50, marking the initial step in the solution preparation. The water then flows through a heater 121, where it is brought to the appropriate temperature for the dialysis process. The temperature of the water is confirmed by a temperature sensor 51, ensuring that the water reaches the desired temperature before it mixes with other components. Connected to the main line 48, an electrolyte concentrate line 52 introduces electrolytes into the system. These electrolytes, stored in a reservoir 53, are delivered into the main line 48 via an electrolyte pump 54. An electrolyte concentrate valve 122 then directs the osmotic agent into the main line 48, where it combines with the purified water.

An osmotic agent line 55 introduces an osmotic agent from a concentrate reservoir 56 into the system. Controlled by an osmotic pump 57, the osmotic agent creates the osmotic gradient required for the dialysis process. An osmotic agent valve 58 then directs the osmotic agent into the main line 48, where it combines with the electrolyte-enriched water.

Following the introduction of the osmotic agent and electrolytes, a mixer 59 ensures thorough blending of the components, producing a homogeneous dialysis solution. The composition of this solution is subsequently assessed by an electrolyte sensor 60 and an osmotic agent sensor 61. These sensors measure the concentrations of electrolytes and osmotic agents, respectively, providing feedback on the solution's composition. A scale 62 is used to weigh a bag 123 containing the mixed solution. This weight measurement is directly connected to the water pump 50, enabling adjustments to the water input based on the solution's total weight.

Feedback loops are employed to adjust the composition based on the measured values from the electrolyte and osmotic agent sensors, as well as the weight measurements from the scale 62. These loops enable dynamic modifications to the flowrates of water, electrolytes, and osmotic agents, ensuring that the dialysis fluid's composition remains within the desired ranges. Once the dialysis fluid's composition is verified to be within the target specifications, it can be directed to storage or immediately used for patient treatment. FIG. 5 shows a configuration featuring both electrolyte and osmotic agent sensors positioned downstream of the concentrate bags and the mixing component. This setup ensures the homogeneity and correct composition of the PDF by monitoring the solution after mixing.

FIG. 5 shows an inline production of peritoneal dialysis fluid (PDF) with sequencing and integration of components to ensure the precise formulation of the dialysis solution. Central to this configuration is the main line 63, which orchestrates the flow and mixing of purified water, osmotic agents, and electrolytes. The process initiates with a purified water reservoir 64, which serves as the source of the solvent for the dialysis solution. Water from this reservoir is pumped through the system by a water pump 65. Subsequently, the water is heated to the appropriate temperature by a heater 66, a step for achieving the optimal reaction conditions for the solution's components. The exact temperature of the water is verified by a temperature sensor 67, guaranteeing that it reaches the desired level before proceeding to mix with the other components.

Connected to the main line 63, an osmotic agent line 68 introduces the osmotic agent into the system from a concentrate reservoir 69. This introduction is controlled by an osmotic pump 70, which establishes the osmotic gradient for the dialysis process. An osmotic agent valve 71 then directs the osmotic agent into the main line 63, ensuring its thorough mixing with the pre-heated water.

Following the osmotic agent, an electrolyte concentrate line 72 adds electrolytes to the mix. These electrolytes, stored in a reservoir 73, are delivered into the main line 63 via an electrolyte pump 74. The addition of electrolytes is vital for mimicking the physiological conditions for effective waste removal and fluid balance during dialysis. An electrolyte concentrate valve 124 then directs the electrolyte concentrate into the main line 63, ensuring its thorough mixing with the pre-heated water.

Downstream in the main line 63, a mixer 75 ensures the integration of the osmotic agent and electrolytes with the water, creating a homogeneous dialysis solution. The composition of this solution is then assessed by an electrolyte sensor 77 and an osmotic agent sensor 76. These sensors are positioned to measure the concentrations of electrolytes and osmotic agents, respectively, providing feedback on the solution's composition.

The system employs feedback loops that connect back to the osmotic pump 70 and electrolyte pump 74, enabling dynamic adjustments to the flowrates of osmotic agents and electrolytes based on the measured values from the sensors. This feedback mechanism ensures that any deviations from the desired concentration ranges of electrolytes and osmotic agents are promptly identified and corrected, maintaining the dialysis solution within optimal composition parameters. Once the composition of the dialysis fluid is confirmed to be within the target specifications by the sensors, it can be directed to storage or immediately utilized for patient treatment.

FIG. 6 shows a system where sensors are placed after the mixing components of the corresponding concentrate bags. In FIG. 6, the electrolytes concentrate is upstream of the osmotic agent concentrate. It is to be appreciated that this order can be reversed (e.g., as shown and described according to FIG. 7 below). This arrangement allows for the precise metering of each concentrate based on its specific sensor feedback before the components are combined. The configuration is centered around the main line 78, which facilitates the sequential addition and mixing of purified water, electrolytes, and osmotic agents to achieve the desired solution composition. The sequence begins with a purified water reservoir 79, from which water is drawn into the system by a water pump 80. Following this, the water is heated to the appropriate temperature by a heater 81, a step that prepares the water for optimal mixing with the solution's other components. The temperature of the water is then verified by a temperature sensor 82, ensuring it has reached the level before proceeding to the next stages of mixing.

Connected to the main line 78, an electrolyte concentrate line 83 introduces electrolytes into the system from a reservoir 84. The delivery of electrolytes into the main line 78 is managed by an electrolyte pump 85. The addition of electrolytes at this stage prepares the solution for the initial mixing process. An electrolyte concentrate valve 125 then directs the electrolyte concentrate into the main line 78, ensuring its thorough mixing with the pre-heated water.

Following the introduction of electrolytes, a first mixer 86 ensures their thorough integration with the pre-heated water. Immediately after this mixing process, an electrolyte sensor 87 assesses the electrolyte concentration in the solution, providing a preliminary check on the solution's composition.

Downstream from the electrolyte integration, an osmotic agent line 88 adds the osmotic agent to the mix from a concentrate reservoir 89. This addition is regulated by an osmotic pump 90, which precisely controls the flow of the osmotic agent into the main line 78 through an osmotic agent valve 91. The introduction of the osmotic agent at this stage is pivotal for establishing the osmotic gradient for the dialysis process.

After the osmotic agent is introduced, a second mixer 126 ensures its complete blending with the previously mixed electrolyte-enriched water, creating a homogeneous dialysis solution. The final composition of this solution is then assessed by an osmotic sensor 127, which measures the osmotic agent concentration, providing feedback on the solution's readiness for dialysis treatment.

The system employs feedback loops that connect back to both the osmotic pump 90 and electrolyte pump 85, enabling adjustments to the flowrates of osmotic agents and electrolytes based on the measured values from the sensors. This mechanism allows for dynamic modifications to the solution's composition, ensuring that the concentrations of electrolytes and osmotic agents remain within the desired ranges for effective dialysis treatment. Once the composition of the dialysis fluid is verified to meet the target specifications, it can be directed to storage or used immediately for patient treatment.

FIG. 7 shows the system designed without positioning constraints for the sensor or sequencing of the osmotic and electrolyte components. This highlights the adaptability of sensor placement in achieving accurate solution composition, even when using refractometer sensors that are sensitive to both electrolyte and osmotic agent solutions. A main line 92 enables integration of purified water, osmotic agents, and electrolytes to formulate the dialysis solution. The process initiates with a purified water reservoir 93, from which water is drawn by a water pump 94. The water then passes through a heater 128, where it is brought to a temperature that is suitable for mixing with the solution's other components. The accuracy of the water's temperature is confirmed by a temperature sensor 95, ensuring it reaches an optimal level for the next phase of the process.

Following the temperature adjustment, an osmotic agent line 96 introduces the osmotic agent into the system from a concentrate reservoir 97. This introduction is regulated by an osmotic pump 98, which precisely controls the flow of the osmotic agent into the main line 92 through an osmotic agent valve 99. The timely addition of the osmotic agent is key for creating the osmotic gradient needed for the dialysis process. After the osmotic agent's entry into the main line 92, a first mixer 106 ensures its thorough blending with the pre-heated water. Subsequently, an osmotic agent sensor 108 evaluates the concentration of the osmotic agent in the mixture, providing an initial assessment of the solution's composition.

Next, an electrolyte concentrate line 100 adds electrolytes to the mix from a reservoir 102. The delivery of electrolytes into the main line 92 is managed by an electrolyte pump 104. An electrolyte concentrate valve 129 then directs the electrolyte concentrate into the main line 92, ensuring its thorough mixing with the pre-heated water.

Following the introduction of electrolytes, a second mixer 110 facilitates their complete integration with the previously mixed osmotic agent-enriched water, producing a uniform dialysis solution. The final composition of this solution is then assessed by an electrolyte sensor 112, which measures the electrolyte concentration, offering feedback on the solution's readiness for dialysis treatment.

The system incorporates feedback loops that connect back to both the osmotic pump 98 and electrolyte pump 104, enabling the adjustment of flowrates based on the measured values from the sensors. This feedback mechanism permits dynamic modifications to the solution's composition, ensuring that the concentrations of osmotic agents and electrolytes stay within the targeted ranges for effective dialysis treatment. Once the composition of the dialysis fluid is confirmed to meet the specified requirements, it can be directed to storage or immediately utilized for patient treatment.

The system employs a variety of sensors to measure the composition of the produced fluid accurately. An electrical conductivity sensor measures the conductivity of the fluid, providing data on electrolyte concentration. A polarimetric sensor can be used to verify the composition of osmotic agents like dextrose without adjusting the metering. That is, one or more additional sensors can be included in the system to provide redundancy in the verification of the PD fluid, with the sensors not contributing to the metering of the fluids. One non-limiting list of sensors for osmotic and electrolyte measurement includes but is not limited to Osmotic Agent Sensors: Polarimetric (optical), Refractometer (optical), Non-enzymatic (electrochemical), Infrared absorption (optical), Spectrophotometer (optical). Electrolyte Sensors: Conductivity (electrochemical), ISE-Ion Selective Electrodes (electrochemical), Refractometer (optical), Spectrophotometer (optical), Polarimetric (optical).

Control Flow Loop: The system's control mechanism is designed around a negative feedback loop for maintaining the desired composition of the PD fluid. The loop adjusts the proportions of concentrate and water based on real-time sensor data, ensuring the fluid's conductivity and osmotic properties match prescribed targets. The incorporation of a positive feedback loop was considered but deemed unfeasible, as the system's goal is to minimize error signals towards target values, necessitating the exclusive use of negative feedback mechanisms for stability and accuracy.

The system is capable of producing PD fluid according to various prescribed recipes, accommodating a range of dextrose concentrations and other formulations to meet diverse patient needs. Below is a Table 1 of non-limiting, representative recipes. It will be understood any variation can be made accurately and efficient using the systems and methods described herein.

TABLE 1

| | Dextrose | Electrolytes | | Osmolality |
|---|---|---|---|---|
| Recipe Type | (%) | (mEq/L) | pH Range | (mOsm/L) |
| Low Dextrose | 1.5-2.5 | Na+ 132-154, | 5.0-7.0 | 250-350 |
| | | Cl− 96-118 | | |
| Medium | 2.6-3.5 | Na+ 132-154, | 5.0-7.0 | 350-450 |
| Dextrose | | Cl− 96-118 | | |
| High Dextrose | 3.6-4.5 | Na+ 132-154, | 5.0-7.0 | 450-550 |
| | | Cl− 96-118 | | |
| Bicarbonate- | N/A | Na+ 132-154, | 7.0-8.0 | 250-350 |
| Based | | HCO3− 25-40 | | |
| Amino Acid | N/A | Amino acids 1.0- | 6.5-7.5 | 300-400 |
| | | 1.5%, Na+ | | |
| | | 132-154 | | |

The system is designed to accommodate a wide range of dialysate formulations, as detailed in Table 2. The system can utilize various osmotic agents in addition to, or in place of, dextrose, including glucose, icodextrin, or amino acids. Different salts such as lactate or acetate salts can replace chloride salts for sodium, magnesium, and calcium. The system accommodates buffers like bicarbonate, acetate, or lactate buffer for maintaining the pH of the peritoneal dialysate. For hypokalemic patients, potassium chloride can be included in the dialysate formulation.

TABLE 2

| Component | Concentration | |
|---|---|---|
| Sodium chloride | 132-134 | mmol/L |
| Calcium chloride | 1.25-1.75 | mmol/L |
| dehydrate | | |
| Magnesium chloride | 0.25-0.75 | mmol/L |
| hexahydrate | | |
| Sodium Lactate | 35-40 | mmol/L |
| Dextrose (D- | 0.55-4.25 | g/dL |

| | |
|---|---|
| glucose) | |
| monohydrate | |
| pH | 5-6 |
| Osmolality | 346-485 |
| | (hypertonic) |

In any embodiment, the concentrate sources can contain one or more osmotic agents and ion concentrates, either as a single source or multiple sources for different components. In any embodiment, the concentrate pump(s) control the addition of concentrated solutions to the peritoneal dialysate generation flow path, with the capability for individual control over each component's movement into the flow path.

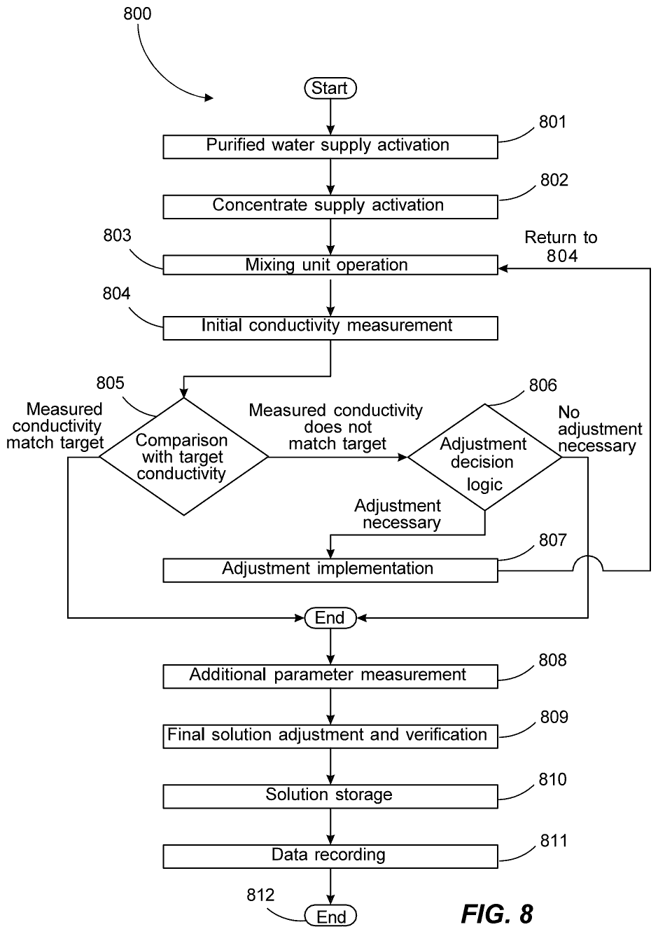

FIG. 8 shows one, non-limiting flow chart for the negative feedback loop in the creation of dialysis fluid. This flow chart provides a negative feedback loop method 800 for creating dialysis fluid, highlighting the detailed steps and considerations that ensure the solution's composition and suitability for peritoneal dialysis treatment.

In step 801, the purified water supply activation, the system initiates the flow of water towards the mixing unit. In some embodiments, the flowrate can be set initially based on a predefined setting. In some embodiments, prior to step 801 or as part of step 801, the system can check and initialize to ensure all components are operational and sterile.

In step 802, the concentrate supply activation, flow of concentrate from the concentrate containers is enabled, allowing the flow of electrolyte, glucose, bicarbonate concentrates, or combinations thereof, towards the mixing unit. Initial flowrates for each concentrate are based on standard formulations.

In step 803, the mixing unit operation, the mixing unit combines purified water and concentrates. This step 803 is controlled to ensure thorough mixing, utilizing separate channels and mixing mechanisms to achieve a homogeneous solution.

In step 804, the initial conductivity measurement, before the full mixing process, an initial conductivity measurement is taken to establish a baseline. This step 804 helps the system obtain information for the starting point of the solution's composition.

In step 805, the comparison with target conductivity, (Mixing Process), the system's controller can be programmed to compares the measured conductivity with the target conductivity values predetermined for the desired dialysis fluid composition.

In step 806, the adjustment decision logic, if the measured conductivity is within acceptable range of the target, the process moves to additional parameter measurement. If the measured conductivity deviates from the target, the system calculates the adjustments in concentrate and water proportions.

In step 807, the conductivity adjustment implementation, the controller adjusts the flowrates of water and/or concentrates based on the calculated needs, increasing or decreasing specific component flows to correct the solution's composition. The system then cycles back to Step 804 for re-measurement and reassessment.

In step 808, the additional parameter measurement, sensors measure other parameters of the solution, such as pH, temperature, and osmolality. These parameters ensure the solution's overall suitability for dialysis treatment.

In step 809, the final adjustment and verification, based on the additional parameter measurements, final adjustments are made to the solution to ensure all aspects of its composition meet the specified criteria for effective and safe dialysis treatment.

In step 810, the solution storage and readiness, the finalized dialysis solution is directed to a storage unit, where it is kept until needed for dialysis treatment. This step 810 ensures the solution is readily available and maintains its composition.

In step 811, the historical data recording and system optimization, the controller records all relevant data from the process, including initial and adjusted conductivity measurements, flowrates, and adjustments made. This historical data is used for continuous learning and system optimization, improving the accuracy and efficiency of future solution preparations.

Upon completion of the process, at step 812, the system either enters a standby mode awaiting the next operation or resets for a new cycle, depending on the operational protocol.

In the design of the peritoneal dialysis fluid production system, a plurality of negative feedback loops can be used to ensure the solution's composition meets specific compositional and therapeutic requirements. Each feedback loop can be responsible for adjusting a particular parameter of the dialysis solution, such as conductivity, pH, temperature, and osmolality, based on predefined target ranges that are for the efficacy and safety of the treatment.

For example, in one non-limiting embodiment, the system can used multiple negative feedback loops dedicated to adjusting electrolyte and glucose concentrations, ensuring they fall within the optimal ranges for dialysis solutions as outlined. In some embodiments, the system can set a target so that it falls within an acceptable tolerance beyond a target. For example, in some embodiments, a threshold may be set with a margin for error of +/−a given percentage such as, but not limited to, 5% (e.g., +/−2.5%). For example, for electrolytes, the system can target sodium chloride concentrations between 132-134 mmol/L, calcium chloride dehydrate at 1.25-1.75 mmol/L, and magnesium chloride hexahydrate at 0.25-0.75 mmol/L. If initial measurements indicate deviations from these targets, the electrolyte feedback loop adjusts the flow of respective electrolyte concentrates into the mixing unit to correct the imbalance. Similarly, for glucose concentration, which is important for achieving the desired osmolality and providing the osmotic gradient for dialysis, the system can aim for a range, for example, of 0.55-4.25 g/dL as indicated by dextrose (D-glucose) monohydrate levels. The glucose feedback loop monitors the solution's glucose concentration and can adjust the dextrose concentrate flow to maintain it within this therapeutic range. These adjustments are for ensuring the dialysis solution effectively removes waste products and excess fluid from the patient's bloodstream while maintaining electrolyte balance and preventing glucose-related complications and be used in concert and orchestrated together using multiple and overlapping negative feedback loops.

In addition to composition, a conductivity feedback loop can adjust the electrolyte concentration, aiming for a target range, for example, of 12.15 mS/cm to 14.00 mS/cm, If the initial conductivity measurement falls outside this range, the loop responds by altering the flowrates of water and electrolyte concentrates to bring the solution back within the desired conductivity parameters. Similarly, the pH feedback loop can be used to ensure the solution's pH stays within a range, for example, of 5.0 to 7.0, adjusting the amounts of bicarbonate or lactate buffer as needed. This is important for preventing irritation of the peritoneal membrane and ensuring the comfort and safety of the patient. For example, the temperature feedback loop maintains the solution's temperature between 35° C. to 37° C., closely mimicking the human body's natural temperature to ensure patient comfort during the dialysis process. If the solution's temperature deviates, the loop activates heating or cooling mechanisms to correct it. An osmolality feedback loop can also be used to keep the solution's osmolality within the range, for example, of 346 to 485 mOsm/L, using measurements of glucose concentration as a primary adjustment factor. This range can be used for creating the correct osmotic gradient needed for effective waste and excess fluid removal from the blood. By employing these feedback loops, the system can dynamically adjust the dialysis solution's composition in response to real-time measurements, ensuring that the final product consistently meets the targeted composition ranges for optimal patient treatment.

In one example, concentrates solutions can be diluted with purified water in a ratio 1:16.2 to produce PDF.

In another example, flowrates can range from 400 to 600 ml/min. Patient fill volumes are typically of 300-3000 mL, for a maximum of 18-21 L within a single night therapy. Therefore, PDF flowrates lower than 50-100 mL/min would require a too long production time. Instead, the higher limit for PDF flowrates is related to the pressures that the system can withstand and PDF composition accuracies. A reasonable upper limit could be 1-2 L/min.

In certain embodiments, temperature sensors can be positioned next to the electrolyte and osmotic agent sensors so that a temperature compensation can be implemented. The temperature of the purified water and not the temperature of the concentrate solutions can be controlled. Mixing temperatures can range, for example, from 5° C. to 37° C. to avoid cooling down the solutions prior to perform the patient fill.

In certain embodiments, target conductivity values and target degree of rotation values will become reference signals in one or more negative feedback loops. In certain non-limiting examples, a target conductivity can be 12.15 mS/cm, 12.00 mS/cm and 11.60 mS/cm for certain dialysis solutions having low, medium and high dextrose concentration respectively

The order of ingredients can be important for electrolytes and the osmotic agent. In particular, a concentrate's variability can be checked with sensors and targeting a range of flowrates to cover variability. Gravimetric scales can be utilized as a virtual flow meter by discerning differences in weight over different time intervals.

In certain embodiments, simultaneous delivery of three streams, emphasizing the order of delivery-starting with water followed by the electrolytes and then the osmotic agents, but going to drain can be used. In other embodiments, sensors can take control and use the obtained measurement to adjust for variability in the concentrates.

One skilled in the art will understand that various combinations and/or modifications and variations can be made in the described systems and methods depending upon the specific needs for operation. Various aspects disclosed herein may be combined in different combinations than the combinations specifically presented in the description and accompanying drawings. Moreover, features illustrated or described as being part of an aspect of the disclosure may be used in the aspect of the disclosure, either alone or in combination, or follow a preferred arrangement of one or more of the described elements. Depending on the example, certain acts or events of any of the processes or methods described herein may be performed in a different sequence, may be added, merged, or left out altogether (e.g., certain described acts or events may not be necessary to carry out the techniques). In addition, while certain aspects of this disclosure are described as performed by a single module or unit for purposes of clarity, the techniques of this disclosure may be performed by a combination of units or modules associated with, for example, a medical device.

What is claimed is:

1. A system for inline production of peritoneal dialysis fluid, comprising:
a mixing section for combining concentrate and water
wherein the mixing section comprises a static mixer,
wherein the mixer is configured to mix the concentrate and the water without any moving parts;
an electrolyte sensor for measuring a produced fluid; and
a controller programmed to adjust proportions of concentrate and water based on the measurements by the electrolyte sensor, thereby forming a peritoneal dialysis solution.

2. The system of claim 1, wherein the controller is further programmed to perform real-time adjustments of the concentrate and water proportions during production of the peritoneal dialysis solution.

3. The system of claim 1, wherein the sensor is an electrical conductivity sensor.

4. The system of claim 1, wherein the mixing section includes separate channels for the concentrate and the water.

5. The system of claim 1, wherein the mixer is configured to mix the concentrate and the water without any moving parts.

6. The system of claim 1, wherein the controller is programmed to store historical data of conductivity measurements and adjustments made.

7. The system of claim 1, further comprising a user interface for displaying current conductivity measurements and adjustments made by the controller.

8. The system of claim 1, wherein the concentrate comprises at least one of:
dextrose, electrolytes, and bicarbonate.

9. The system of claim 1, wherein the water is purified or sterilized water.

10. The system of claim 1, further comprising one or more negative feedback loops for enhanced precision in solution preparation.

11. The system of claim 10, wherein each negative feedback loop includes additional sensors for measuring other parameters of the peritoneal dialysis solution.

12. The system of claim 11, wherein the other parameters include pH, temperature, and osmolality.

13. The system of claim 1, wherein the system is configured for use in both clinical and home settings.

14. A system for inline production of peritoneal dialysis fluid, comprising:
a mixing unit for combining concentrate and water;
wherein the mixing unit comprises a static mixer;
a sensor for measuring conductivity of produced fluid; and
a controller programmed to adjust proportions of concentrate and water based on the conductivity measurements, thereby forming a dialysis solution.

15. The system of claim 14, wherein the controller is further programmed to perform real-time adjustments of the concentrate and water proportions during production of the dialysis solution.

16. The system of claim 14, wherein the mixing unit includes separate channels for the concentrate and the water.

17. The system of claim 14, further comprising a storage unit for prepared dialysis solution.

18. The system of claim 14, further comprising a user interface for displaying current conductivity measurements and adjustments made by the controller.

19. The system of claim 14, wherein the concentrate comprises at least one of:
glucose, electrolytes, and bicarbonate.

20. The system of claim 14, further comprising one or more negative feedback loops for enhanced precision in solution preparation.
